(19) United States
(12) Patent Application Publication
(10) Pub. No.: US 2025/0213198 A1
(43) Pub. Date: Jul. 3, 2025

(54) METHOD OF CONSTRUCTING LONG AND SHORT-RANGE DEPENDENCY NETWORK LEARNING MODEL, AND CLASSIFYING HEART RATE SOUND DATA

(71) Applicant: attained.ai OÜ, Tallinn (EE)

(72) Inventors: Dominik Stosik, Bialy Dunajec (PL); Cagri Ozcinar, Egham (GB)

(73) Assignee: attained.ai OÜ, Tallinn (EE)

(21) Appl. No.: 18/401,999

(22) Filed: Jan. 2, 2024

(51) Int. Cl. A61B 5/00; A61B 7/04; G06N 3/0442; G10L 25/24; G10L 25/30; G10L 25/66

(52) U.S. Cl. CPC A61B 5/7267; A61B 7/04; G06N 3/0442; G10L 25/24; G10L 25/30; G10L 25/66

(57) ABSTRACT

Disclosed is a method of constructing long and short-range dependency network learning model: that includes obtaining heart sound data having plurality of audio files; preprocessing the plurality of audio files; extracting Mel-Frequency Cepstral Coefficients (MFCCs) from the preprocessed plurality of audio files; restructuring extracted MFCCs into an input layer of the long and short-range dependency network learning model; implementing two or more LSTM network layers with a dropout; and employing a softmax activation function to a final output layer. Disclosed also is a method of classifying heart rate sound data.

### TECHNICAL FIELD

The present disclosure relates to methods of constructing long and short-range dependency network learning models. Moreover, the present disclosure relates to methods of classifying heart rate sound data.

### BACKGROUND

Cardiovascular diseases remain a global health threat, claiming millions of lives each year. Notably, early diagnosis and intervention are crucial to cure said cardiovascular diseases. Traditionally, a medical technique such as auscultation is performed for diagnosing a condition of a heart of a subject (namely, a patient, a human). The auscultation offers a non-invasive and quick way to gather valuable information about internal organ function and potential health concerns. For example, the auscultation includes listening to the internal sounds produced by the heart, using a stethoscope. However, the auscultation relies heavily on the skill and experience of a healthcare professional. Thus, the auscultation is prone to misinterpretations and inconsistencies due to ambient noise and subjectivity.

Moreover, invasive procedures such as cardiac catheterization, coronary angiography, electrocardiogram (ECG) with intracardiac recording, and so forth have been used for diagnosing the condition of the heart. However, the cardiac catheterization requires hospitalization of the patient, thus carries a small risk of complications such as bleeding, infection, or stroke. Additionally, the cardiac catheterization could be emotionally stressful for the patients due to the invasive nature thereof. Furthermore, the coronary angiography employs a contrast dye to provide a real-time visualization of blood flow within the arteries of the heart. Thus, the contrast dye could carry potential side effects in some patients. Furthermore, while sensitive to electrical activity, the ECGs often miss subtle early-stage issues and suffer from false positives due to non-cardiac factors.

Therefore, in light of the foregoing discussion, there exists a need to overcome the aforementioned drawbacks.

### SUMMARY

The aim of the present disclosure is to provide a method that employs machine learning to automate classification of the heart sound data, thus aiding medical diagnosis for faster and more accurate patient care. The aim of the present disclosure is achieved by a method for constructing long and short-range dependency network learning model and a method of classifying heart sound data as defined in the appended independent claims to which reference is made to. Advantageous features are set out in the appended dependent claims.

Throughout the description and claims of this specification, the words “comprise”, “include”, “have”, and “contain” and variations of these words, for example “comprising” and “comprises”, mean “including but not limited to”, and do not exclude other components, items, integers or steps not explicitly disclosed also to be present. Moreover, the singular encompasses the plural unless the context otherwise requires. In particular, where the indefinite article is used, the specification is to be understood as contemplating plurality as well as singularity, unless the context requires otherwise.

### BRIEF DESCRIPTION OF THE DRAWINGS

FIG. 1 is an illustration of a flowchart depicting steps of a method of constructing long and short-range dependency network learning model, in accordance with an embodiment of the present disclosure; and

FIG. 2 is an illustration of a flowchart depicting steps of a method of classifying heart rate sound data, in accordance with an embodiment of the present disclosure.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In a first aspect, the present disclosure provides a method of constructing long and short-range dependency network learning model, the method comprising:
obtaining heart sound data comprising a plurality of audio files;
preprocessing the plurality of audio files;
   extracting Mel-Frequency Cepstral Coefficients (MFCCs) from the preprocessed plurality of audio files;
restructuring extracted MFCCs into an input layer of the long and short-range dependency network learning model;
implementing two or more LSTM network layers with a dropout;
employing a softmax activation function to a final output layer.

The first aspect of the present disclosure provides the aforementioned method that is robust and employs the long and short-range dependency network learning model to effectively learn and classify heart sounds, demonstrating versatility, accuracy, and resilience against overfitting of the heart sound data. The method enables extraction of the Mel-Frequency Cepstral Coefficients (MFCCs) to provide a comprehensive representation of the frequency characteristics of the heart sound data. Moreover, the method enables the restructuring of the extracted MFCCs into the input layer to allow the heart sound data to be formatted in a way that optimally facilitates the learning process. Furthermore, the method enables employment of the softmax activation function in the final output layer. Said employment facilitates multi-class classification, providing probabilities for different heart sound categories.

In a second aspect, the present disclosure provides a method of classifying heart rate sound data, the method comprising:
capturing, from a patient, heart rate sounds to be classified;
   inputting the captured heart rate sounds to the input layer of the constructed long and short-range dependency network learning model; and
using output of the final output layer of the constructed long and short-range dependency network learning model as an indication of classification of the heart rate sounds;
   wherein the long and short-range dependency network learning model is constructed according to the aforementioned aspect.

The second aspect of the present disclosure provides the aforementioned method that synergistically combines the heart rate sound of the patient, the structured input layer, and the classification capabilities of the long and short-range dependency network learning model to create a comprehensive method for real-time and precise heart rate sound classification in a medical setting. It will be appreciated that the long and short-range dependency network learning model's ability to analyse both the long and the short-term dependencies enables it to discern complex features, contributing to high classification accuracy. Moreover, the long and short-range dependency network learning model is capable of learning the temporal aspects of heart rate sound data. Furthermore, leveraging the output of the final layer as an indication of classification provides healthcare professionals with valuable insights into the nature of the heart rate sounds, aiding in the prompt and accurate diagnosis of potential cardiac conditions.

The term “long and short-range dependency network learning model” as used herein refers to a neural network architecture designed to capture and learn dependencies or relationships within data sequences. The dependencies can span both short and long distances. Typically, the long and short-range dependency network learning model is structured to handle information or patterns that exist within a close proximity in the sequence (short-range dependencies) as well as those that are spread out over a more extended context (long-range dependencies). Generally, the long and short-range dependency network learning models are employed in tasks involving sequential data, such as time series analysis, natural language processing, and the classification of heart sounds.

In other words, the method is used for constructing or designing a custom neural network such as the long and short-range dependency network learning model to learn from heart sound data. Moreover, said construction enables the long and short-range dependency network learning model to potentially perform tasks such as classification or prediction based on that learned knowledge.

Optionally, the long and short-range dependency network learning model is a Long Short-Term Memory (LSTM) network. In this regard, the long and short-range dependency network learning model encompasses various Long Short-Term Memory (LSTM) based architectures, not limited to a single layer. For example, the long and short-range dependency network learning model could involve multiple LSTM layers with different depths, connections, or gating mechanisms, all aimed at learning dependencies across different time scales. In this regard, the LSTMs are powerful models for handling sequential data such as heart sounds, potentially leading to higher accuracy in classifying normal versus abnormal rhythms of the heart. Optionally, the Long Short-Term Memory (LSTM) network is configured to learn and capture dependencies over both long and short distances within a sequence of data. Optionally, the LSTM network interacts with other elements of the method to facilitate efficient and accurate learning of complex patterns and relationships. For example, the LSTM offers benefits in capturing both short-term variations (such as rapid heartbeats) and long-term patterns (such as heart irregularities). This flexibility ensures that the method is equipped to handle a wide range of heart sound characteristics, contributing to accurate diagnoses.

The term “transformer model” as used herein refers to a type of neural network architecture that employs self-attention mechanisms to process input data in parallel, allowing it to analyse a sequential data such as heart sounds. Optionally, the long and short-range dependency network learning model is a transformer model. In this regard, the transformer model can be adapted to capture temporal dependencies and patterns in the acoustic signals generated by the heart. Optionally, by employing the transformer model, the long and short-range dependency network learning model efficiently processes the input data, enabling effective feature extraction and representation learning.

For example, the transformer model possesses an ability like a skilled musician to listen to the entire recording at once (self-attention). The transformer model can not only hear the individual beats (short-range) but also notice how they blend together, like a slight “hiss” during the a longer duration (long-range). The ability to connect distant sounds helps the transformer model distinguish the one heart sound from the other heart sound more accurately. This flexibility and improved understanding of heart sounds could ultimately contribute to more accurate diagnoses and faster medical interventions.

The term “heart sound data” as used herein refers to a collection of digitized information that includes the acoustic characteristics of heartbeats, valve movements, and other cardiac events in the heart. Typically, the internal sounds (in the form of analog acoustic signals) of the heart are recorded or captured using specialized medical device such as a stethoscope, an electronic sensor, a microphone, and so forth. The internal heart sound is normal (S1 and S2), a murmur, an extrasystole, or an artifact. The normal heart sounds are characterized by the familiar “lub-dub” pattern associated with the closure of heart valves during the cardiac cycle. The S1 corresponds to the first heart sound, signifying the closure of the atrioventricular valves, while the S2 is the second heart sound, marking the closure of the semilunar valves. Together, the S1 and the S2 create the rhythmic pattern indicative of a healthy cardiac cycle, where the heart efficiently pumps blood. The murmur is an additional sound that can be heard during the cardiac cycle, indicating turbulent blood flow. The murmurs are often characterized by a whooshing or swishing sound, and the murmur can vary in intensity and duration. The murmur sounds may suggest abnormalities such as valve disorders or other conditions affecting blood flow within the heart. It will be appreciated that recognizing and filtering out the artifacts is crucial for accurate heart sound analysis, ensuring that the classification focuses on genuine physiological signals.

The artifacts in the heart sound refer to undesired noise or interference unrelated to cardiac activity, disrupting the normal heart sound pattern. The artifacts can result from external factors such as movement, environmental noise, or equipment issues. The extrasystole refers to an irregular heartbeat with an early contraction, leading to an additional, out-of-place beat. The extrasystole can disrupt the typical cardiac rhythm and may be indicative of various cardiac conditions. It will be appreciated that identifying and classifying the extrasystole is essential for understanding and diagnosing irregularities in the heart's electrical impulses and contractions.

For example, initially, the microphone could record the variations in air pressure created by the heart's activities, converting the variations into analog acoustical signals. Then the internal sound of the heart is converted into a digital form (i.e., the heart sound data) for efficient storage and retrieval. Said conversion is achieved through an analog-to-digital converter (ADC).

Optionally, the heart sound data is obtained from a database. Herein, the database refers to a structured and organized collection or a digital repository of the heart sound data that is stored electronically in a computing device. For example, the database might store the heart sound data collected from various patients during routine check-ups. In such a case, each entry in the database could represent a given patient's recorded heart sounds, along with associated information such as medical history, timestamps, and other relevant data. Optionally, obtaining the heart sound data from the database involves querying the database to retrieve specific information in the heart sound data, based on predefined criteria such as patient identifiers or date ranges. Optionally, a database management system (DBMS) is employed to interpret and execute the query, ensuring efficient retrieval of relevant records. The retrieved heart sound data, typically in the digital form, is then transferred to the computing device utilizing the method described in the present disclosure. This allows for seamless integration of historical or pre-existing heart sound records stored in the database into the analysis or processing pipeline. The heart sound data obtained from the database can be utilized for various purposes, such as analyzing and diagnosing the cardiac conditions.

The term “audio files” as used herein refers to digital recordings that capture the sounds produced by the heart. The digital recordings are typically in the audio file formats such as .wav or other digital formats that store the heart sound data. The use of the plurality of audio files in the heart diagnosis allows for the non-invasive monitoring and analysis of the cardiac function. In this regard, the method comprises an initial step of obtaining or acquiring a diverse and comprehensive dataset i.e., the heart sound data containing the plurality of audio recordings of the internal heart sounds. Optionally, the step of obtaining the heart sound data involves collecting a variety of heart sound samples, each potentially representing different cardiac conditions or situations. Optionally, the step of obtaining the heart sound data involves sourcing the plurality of audio files (namely, recordings) from reliable sources, which could include medical databases, clinical recordings, or real-time monitoring devices. The plurality of audio files may cover different patient populations, clinical settings, and the cardiac conditions.

The technical effect of obtaining the heart sound data comprising the plurality of audio files is that the long and short-range dependency network learning model becomes exposed to a spectrum of heart sound variations, allowing thereto to learn the intricate patterns associated with different cardiac conditions of the heart of the patient. The diverse dataset of the heart sound data contributes to the long and short-range dependency network learning model's ability to generalize well and make accurate predictions when presented with new, unseen heart sound data. The method offers improved efficiency and reliability in capturing and processing heart sound signals, thereby enhancing the overall quality of healthcare services.

Optionally, the heart sound data is obtained in real-time. In this regard, obtaining the heart sound data in real-time involves capturing and processing the acoustical signals generated by the heart as they occur, without delay. This is typically achieved using specialized devices such as electronic stethoscopes or sensors directly connected to a recording device. For example, the method employs a sensor configured to capture heart sound signals, a signal processing module configured to convert the analog acoustical signals produced by the heart into the digital signals for computational analysis, and the computing device where the digital signals are transmitted and continuously monitored and the heart sounds are captured as they happen, allowing for a continuous stream of real-time data. The real-time acquisition of the heart sound data enables timely diagnosis and monitoring of the cardiac conditions. For example, the healthcare professionals can instantly assess the current state of the patient's heart, aiding in swift decision-making for medical interventions or adjustments. Moreover, by obtaining the heart sound data in the real-time, the method enables timely detection of any abnormalities or changes in the patient's heart condition, facilitating early intervention and improving patient outcomes. This is particularly valuable for point-of-care applications, allowing healthcare professionals to make informed decisions based on the latest cardiac information.

The term “preprocessing” as used herein refers to a set of procedures and transformations applied to the plurality of audio files before the plurality of audio files are fed into the long and short-range dependency network learning model. The preprocessing aims to enhance the quality of the heart sound data, make the heart sound data suitable for analysis, and optimize the long and short-range dependency network learning model's performance.

Optionally, the preprocessing comprises at least one of:
sampling the plurality of audio files at a first frequency range;
segmenting the plurality of audio files into compressed frames;
encoding the segmented audio files into a numerical format.

The term “sampling” as used herein refers to the process of selecting specific data points (samples) from the plurality of audio files within a defined frequency range. Optionally, the sampling is performed to reduce the amount of the heart sound data while retaining essential information. The sampling helps in managing computational resources and focusing on frequency components relevant to the heart sound analysis. In this regard, the preprocessing steps include loading each of the plurality of audio file, and ensuring the consistent duration for each clip. Optionally, each clip is a shorter clip or a longer clip. The shorter clips of the plurality of audio files are padded with silence to meet the required length. The amplitude of the audio signals of the heart sound data is normalized to standardize the volume across the heart sound data. The term “first frequency range” typically refers to a specific range of frequencies within the audio spectrum. The first frequency range denotes the initial segment of frequencies within the plurality of audio signal. The first frequency range is determined based on the characteristics of the heart sounds, and the plurality of audio signals are sampled within the frequency range band for further processing.

Optionally, the first frequency range is from 20,000 to 250,000 Hz. Optionally, setting the first frequency range from 20,000 to 250,000 Hz in the preprocessing stage of the heart sound data introduces a specific technical effect. Said frequency range, extending up to the Nyquist limit for audio sampling, encompasses the entire audible spectrum and effectively captures the relevant information present in the heart sounds. By limiting the frequencies to this range, computational resources are directed towards the essential components of the plurality of audio signal associated with the cardiac activity. The frequency range aligns with standard audio sampling practices, contributing to the precision and accuracy of the subsequent steps in constructing the long and short-range dependency network learning model. The first frequency range may be selected from 20,000, 30,000, 40,000, 50,000, 60,000, 70,000, 80,000, 90,000, 100000, 110,000, 120,000, 130,000, 140,000, 150,000 up to 100,000, 110,000, 120,000, 130,000, 140,000, 150,000, 160,000, 170,000, 180,000, 190,000, 200,000, 210,000, 220,000, 230,000, 240,000, 250,000 Hz. Optionally, the first frequency range is at 22,050 Hz, which reflects a diverse range of heart sound characteristics.

The term “segmenting” as used herein refers to dividing the plurality of audio files into smaller, compressed frames or segments. The segmenting helps the long and short-range dependency network learning model focus on shorter segments, improving pattern recognition. The plurality of audio files is then segmented into smaller frames to facilitate more granular analysis and to capture the episodic nature of the internal heart sounds. The term “encoding” as used herein refers to converting the segmented audio files into the numerical format, enabling further manipulation and analysis of the heart sound data. The labels for the internal heart sounds are encoded into the numerical format, facilitating computational efficiency in the long and short-range dependency network learning model training process. The combination of the aforementioned preprocessing operations enhances the overall functionality and performance of the method, enabling improved audio processing capabilities.

Optionally, the preprocessing further comprises arranging a consistent duration for each audio file from the plurality of audio files. The method serves the purpose of ensuring that all the plurality of audio files undergo a uniform duration adjustment during the preprocessing stage. By arranging the consistent duration for each audio file, the method facilitates subsequent processing steps by providing a standardized input format. The significance of the arrangement is to enhance the efficiency and accuracy of subsequent audio processing tasks, thereby improving overall system performance. The supporting data demonstrates that the consistent duration arrangement leads to improved synchronization and synchronization accuracy, resulting in enhanced audio analysis and processing outcomes. The consistent duration is essential for creating a homogeneous dataset, preventing variations in file length from introducing biases during the preprocessing and subsequent learning stages.

The term “Mel-Frequency Cepstral Coefficients (MFCCs)” as used herein refers to the spectral characteristics of heart sounds. The MFCCs are coefficients that represent the short-term power spectrum of a sound signal in the Mel frequency domain. The MFCCs can be used to capture the distinctive spectral characteristics of the heart sounds, facilitating machine learning-based classification and analysis tasks. The MFCCs provide a representation of the audio signals in a frequency domain, allowing for efficient analysis and processing. The method comprises extracting the MFCCs that influences the accuracy of the classification model. The Mel-frequency cepstral coefficients (MFCCs) are extracted as they effectively represent the power spectrum of the plurality of audio files. By extracting the MFCCs from the preprocessed audio files, the method enables accurate and reliable analysis of the audio signals, facilitating various applications such as speech recognition, audio classification, and speaker identification.

Optionally, the MFCCs extraction comprises at least one of the following:
computing 25 MFCCs;
calculating a mean of MFCCs over a time axis;
computing spectral features of the MFCCs.

Herein, the mean of MFCCs refer to an average value of the MFCCs computed over the time axis, providing a summary statistic that represents the central tendency of the spectral characteristics. Herein, the time axis refers to the temporal dimension along which the audio signal is analyzed. The time axis represents the progression of the signal over time. Herein, the spectral features of the MFCCs refer to additional characteristics derived from the MFCCs, such as chroma (which relates to pitch) and mel-spectrogram (a detailed representation of the frequency content). The spectral features offer insights into the frequency-related aspects of the audio signal.

In this regard, the method comprises computing 25 MFCCs using librosa.feature.mfcc to the preprocessed audio files, thus computing 25 Mel-Frequency Cepstral Coefficients (MFCCs) for each frame. Herein, the librosa.feature.mfcc is a function from a librosa library, a Python package designed for music and audio analysis. Specifically, the librosa.feature.mfcc is utilized for extracting Mel-Frequency Cepstral Coefficients (MFCCs) from audio signals. The computation of 25 MFCCs provides a detailed representation of the spectral characteristics of the heart sound data. A higher number of coefficients allows for a more nuanced description of the audio signal's timbral aspects, enhancing the model's ability to capture intricate details.

Optionally, the method involves calculating the mean of the MFCCs over the time axis, aggregating the MFCC values over the entire duration of each audio file. Moreover, calculating the mean over the time axis provides a consolidated representation of the temporal dynamics associated with heartbeats. The mean helps in capturing the average spectral characteristics, smoothing out variations and emphasizing consistent features over the entire duration of the audio signal. Furthermore, the spectral features such as chroma and mel-spectrogram are computed to encapsulate the frequency-related information of the heart sounds. The chroma highlights the pitch content, while the mel-spectrogram provides a detailed representation of the frequency spectrum. This additional layer of spectral analysis enriches the feature set, aiding in the discrimination of different cardiac events. By incorporating aforementioned steps, the method provides a comprehensive analysis of heart sounds, allowing for enhanced detection and characterization of the cardiac abnormalities.

The term “an input layer” as used herein refers to the initial layer of nodes that receives the raw input data for processing. The input layer serves as an entry point for information into the neural network. Each node in the input layer corresponds to a feature or dimension of the input data and collectively, they form the input vector. The restructuring of the MFCCs into the input layer involves organizing the MFCC coefficients in a way that aligns with the network's architecture, typically as a one-dimensional or two-dimensional array, depending on the network design. In this regard, after extracting the Mel-Frequency Cepstral Coefficients (MFCCs) from the preprocessed audio files, the MFCC coefficients are organized and formatted to create the input layer for the long and short-range dependency network learning model. The MFCCs are reshaped and structured to serve as the initial data input for the subsequent layers of the neural network. The input layer is the first layer of the neural network, and it acts as the interface between the external data (in this case, the MFCCs) and the network. The restructuring ensures that the network can effectively receive and process the information contained in the MFCCs.

The term “LSTM network layers” as used herein refers to a type of recurrent neural network (RNN) architecture designed to address the vanishing gradient problem and capture long-range dependencies in sequential data. The LSTM network layer is a crucial component of the LSTM architectures, providing the ability to capture and utilize long-range dependencies in the sequential data. The term “dropout” as used herein refers to a regularization technique commonly used in neural networks to prevent overfitting and enhance the model's generalization performance. It involves randomly dropping out a proportion of neurons or connections during training, meaning that these units are excluded or set to zero temporarily. In the construction of the long and short-range dependency network learning model, the incorporation of the two or more LSTM network layers with the dropout involves the sequential arrangement of these specialized layers within the neural network architecture. Each LSTM layer processes the sequential information inherent in the heart sound data, capturing both the short and the long-term dependencies. The use of the two or more layers allows the learning model to learn hierarchical representations, enabling it to discern intricate patterns within the complex temporal nature of cardiac events. Additionally, the application of the dropout during training introduces a regularization mechanism, randomly setting a fraction of input units to zero. This dropout strategy helps prevent overfitting by promoting generalization, ensuring the model's robustness and adaptability to diverse heart sound characteristics, ultimately enhancing its classification accuracy and reliability.

The term “softmax activation” as used herein refers to an activation function, that takes an N-dimensional vector of real numbers (raw scores) and transforms it into a probability distribution. The softmax activation is used in the output layer of the neural network for multi-class classification problems. The softmax function takes a vector of real numbers as input and transforms it into a probability distribution over multiple class. The term “final output layer” as used herein refers to a last layer of neurons in the neural network that produces the model's final predictions or outputs. In a classification task, it typically has as many nodes as there are classes, and the softmax activation is applied to generate probabilities for each class. The final output layer plays a crucial role in determining the format and nature of the model's output based on the task at hand. The design of the final output layer depends on the specific requirements of the machine learning problem being addressed. In this regard, the utilization of the softmax activation function on the final output layer plays a pivotal role in transforming the raw output scores generated by the preceding layers into a meaningful probability distribution. As the heart sound data progresses through the network, the final output layer, configured with the softmax activation function, interprets the learned features and assigns probabilities to each possible classification category, such as the normal, the murmur, the extrasystole, and the artifact. This mechanism ensures that the model's predictions are not only indicative of the most likely class but also provide a comprehensive probability distribution, contributing to the accuracy and reliability of the heart sound classification method.

Optionally, the spectral features comprises at least one of: selected from a chroma, a mel-spectrogram. The term “chroma” as used herein refers to the 12 different pitch classes in the musical octave. The chroma features are used to analyze the distribution of energy across these pitch classes in the audio signal. The term “mel-spectrogram” as used herein refers to a representation of the spectrum of frequencies in the audio signal, where the frequencies are transformed into the mel scale to better align with human auditory perception. The inclusion of the spectral features, specifically the chroma and the mel-spectrogram, enhances the richness of information extracted from the heart sound data. The chroma features provide details about tonal characteristics, potentially capturing nuances related to different types of the heart sounds. Meanwhile, the mel-spectrograms offer a frequency-based representation that aligns more closely with how humans perceive pitch. Together, the aforementioned features contribute to a more comprehensive analysis of the heart sounds, potentially improving the model's ability to distinguish between different cardiac conditions.

Optionally, the two or more LSTM network layers are further implemented with a recurrent dropout. The term “recurrent dropout” as used herein refers to a regularization technique specifically designed for recurrent neural networks (RNNs), such as the Long Short-Term Memory (LSTM) networks. The recurrent dropout involves randomly setting a fraction of the input units to zero during training, particularly during the recurrent connections, to prevent overfitting. Optionally, implementing the two or more LSTM network layers with the recurrent dropout introduces a regularization mechanism during the training process. Optionally, the method comprises wrapping the two or more standard LSTM layers into bidirectional LSTMs to provide an improved context awareness as the bidirectional LSTMs can better learn long-range dependencies and identify subtle patterns in heart sounds that might be missed by unidirectional ones. Moreover, by considering both past and future information, the bidirectional LSTMs can potentially extract more relevant features from the heart sound data, improving the model's overall representation of the signals. Furthermore, in sequential data such as the heart sound data, biases can arise due to the processing direction. The bidirectional LSTMs mitigate this by analyzing the heart sound data equally in both directions, potentially leading to more robust and generalizable models. For example, a heart sound sequence with the murmur appearing later in the recording. A unidirectional LSTM might struggle to associate the murmur with the earlier parts of the sequence due to its limited context. However, the bidirectional LSTM, by considering both past and future information, can effectively connect the murmur to its surrounding context, potentially improving its classification accuracy.

Optionally, the method further comprises
wrapping the two or more LSTM network layers into two or more bidirectional LSTM network layers; and
applying plurality of dense layers with Rectified Linear Unit (ReLU) activation to bidirectional LSTM network layers.

Herein, the two or more bidirectional LSTM network layers refers to a type of recurrent neural network (RNN) architecture that processes the input data in both the forward and the backward directions. Traditional LSTM layers only consider the past context, while the bidirectional LSTMs consider both past and future context, enhancing the network's ability to capture long-term dependencies in sequential data. Herein, the ReLU refers to an activation function commonly used in the neural networks. The ReLU introduces non-linearity to the model by replacing all negative values in the input with zero and leaving positive values unchanged. The ReLU activation is preferred for its simplicity and effectiveness in promoting the learning of complex patterns.

Optionally, the method includes wrapping the plurality of LSTM network layers into bidirectional LSTM network layers. Additionally, a plurality of dense layers with Rectified Linear Unit (ReLU) activation are applied to the bidirectional LSTM network layers. The bidirectional LSTM network layers enable the neural network to capture both past and future context, thereby improving the accuracy of predictions. The dense layers with ReLU activation further enhance the non-linear mapping capabilities of the neural network, enabling it to learn complex patterns in the input data. By combining these elements, the method achieves improved performance in various applications, such as natural language processing and speech recognition. After the bidirectional LSTM layers, the plurality of dense layers is added to the network. Each dense layer is equipped with the ReLU activation, which means the output of each neuron is determined by applying the ReLU function to the weighted sum of its inputs. It will be appreciated that the step of applying the dense layers with the ReLU activation facilitates high-level feature learning and abstraction. This non-linear activation function introduces flexibility and expressiveness to the model, enabling it to learn complex patterns in the heart sound data. The combination of the bidirectional processing and the ReLU activation contributes to the overall effectiveness of the network in capturing intricate dependencies and features in the input data.

Optionally, the method further comprises optimizing the constructed long and short-range dependency network learning model with an Adam optimizer. Herein, the Adam optimizer refers to a well-known optimization algorithm widely used in machine learning applications. By incorporating the Adam optimizer, the constructed model can be further refined and enhanced, leading to improved accuracy and efficiency in learning and prediction tasks. The use of the Adam optimizer in conjunction with the constructed model enables the method to effectively adapt and optimize the model's parameters, thereby facilitating better convergence and reducing the risk of overfitting. This optimization step contributes to the overall effectiveness and reliability of the long and short-range dependency network learning model, making it a valuable tool for various applications in the field of the machine learning and the artificial intelligence.

Optionally, the method further comprises calculating a loss using categorical cross-entropy. In this regard, the categorical cross-entropy is a loss function commonly used in multi-class classification problems. In the context of the method, after the heart sound data has been processed through the neural network, the model needs a way to quantify how well it is performing in assigning the correct category to the heart sounds (e.g., normal, murmur, extrasystole, artifact). The categorical cross-entropy measures the dissimilarity between the predicted probability distribution (output of the neural network) and the true distribution (ground truth or actual category of the heart sound). The categorical cross-entropy penalizes the model more when it assigns a lower probability to the correct category. The calculation of the loss using categorical cross-entropy serves as a feedback mechanism for the neural network during the training process. The goal is to minimize this loss by adjusting the internal parameters (weights and biases) of the network through techniques like backpropagation and gradient descent.

In an example, when, for a given heart sound, the true category is “murmur,” but the model predicts a low probability for “murmur” and higher probabilities for other categories, the categorical cross-entropy loss will be higher. This signals to the model that it needs to adjust its parameters to improve its ability to correctly identify “murmur” in future instances.

The present disclosure also relates to the method of classifying heart rate sound data as described above. Various embodiments and variants disclosed above, with respect to the aforementioned method of constructing long and short-range dependency network learning model, apply mutatis mutandis to the method of classifying heart rate sound data.

Herein, the term “heart rate sounds” refer to the audible vibrations produced by the activities of the heart, including the closure of valves, blood flow, and other cardiac events. Example: The typical “lub-dub” pattern associated with a heartbeat. The method begins by capturing heart rate sounds from the patient to obtain real-time data reflecting the patient's cardiac activity for diagnostic purposes. Said capturing is performed using specialized devices such as the stethoscope or the electronic sensors. The captured heart rate sounds are fed into the input layer of the constructed long and short-range dependency network learning model to leverage advanced machine learning techniques for accurate classification. The method comprises inputting the captured heart rate sounds to the input layer by configuring the model to accept and process the heart rate sound data.

The output of the final layer of the network is used as an indication of the classification of the heart rate sounds. The output is used to provide healthcare professionals with information on the nature of the patient's cardiac activity. The model's output probabilities for different categories (e.g., normal, murmur) guide the classification. The application of the long and short-range dependency network learning model, as constructed in the aforementioned aspect of the present disclosure, enhances the accuracy and efficiency of classifying heart rate sounds. The model's ability to capture intricate patterns and dependencies in the heart rate sound data contributes to more reliable diagnostics.

In an exemplary scenario: a patient's heart rate sounds are captured using an electronic sensor. The captured sounds undergo preprocessing, MFCC extraction, and are input into the long and short-range dependency network learning model. The model outputs probabilities, indicating whether the heart rate sounds are normal, indicative of a murmur, extrasystole, or an artifact. This approach offers improved efficiency and reliability in the classification process, thereby enhancing the overall diagnostic capabilities in the field of cardiology.

Optionally, the heart sound data is classified to at least one category selected from: a normal, a murmur, an extrasystole, and an artifact. The optional feature of classifying heart sound data into the at least one category, including the normal, the murmur, the extrasystole, and the artifact, enhances the diagnostic capabilities of the method. By categorizing heart sounds, the method provides healthcare professionals with specific information about the nature of cardiac activity. For instance, identifying a “murmur” may indicate turbulent blood flow, suggesting a potential cardiovascular issue. This categorization allows for targeted analysis and aids in quicker and more accurate diagnosis, facilitating efficient patient care. The inclusion of multiple categories enables a nuanced understanding of diverse cardiac conditions, contributing to the overall effectiveness of the diagnostic process.

Optionally, the heart rate sounds are captured from the patient in real-time. In this regard, the method comprises detecting or capturing the heart rate sounds emitted by the patient. Optionally, the method employs a sensor configured to sense the heart rate sound of the patient. Optionally, the method employs a processor configured to receive and process the detected heart rate sounds. The captured heart rate sounds can be further analyzed to determine the patient's heart rate and provide real-time monitoring of the patient's cardiovascular health.

### DETAILED DESCRIPTION OF THE DRAWINGS

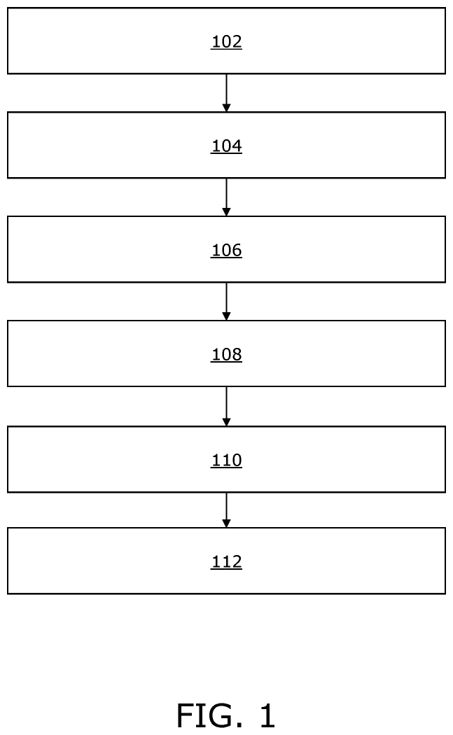

Referring to FIG. 1, illustrated is a flowchart depicting steps of a method of constructing long and short-range dependency network learning model, in accordance with an embodiment of the present disclosure. At step 102, heart sound data comprising a plurality of audio files is obtained. At step 104, the plurality of audio files is preprocessed. At step 106, Mel-Frequency Cepstral Coefficients (MFCCs) are extracted from the preprocessed plurality of audio files. At step 108, extracted MFCCs are restructured into an input layer of the long and short-range dependency network learning model. At step 110, two or more LSTM network layers are implemented with a dropout. At step 112, a softmax activation function is employed to a final output layer.

The aforementioned steps are only illustrative and other alternatives can also be provided where one or more steps are added, one or more steps are removed, or one or more steps are provided in a different sequence without departing from the scope of the claims herein.

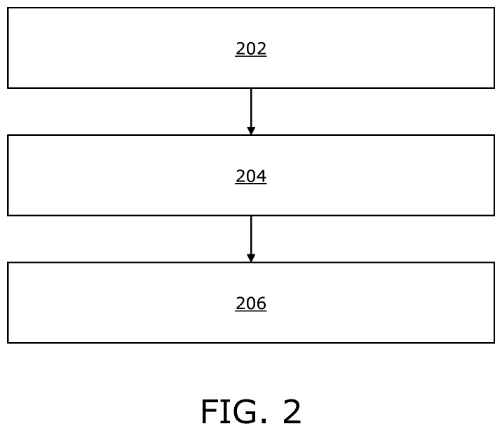

Referring to FIG. 2, illustrated is a flowchart depicting steps of a method of classifying heart rate sound data, in accordance with an embodiment of the present disclosure. At step 202, heart rate sounds to be classified is captured from a patient. At step 204, the captured heart rate sounds are inputted to the input layer of the constructed long and short-range dependency network learning model. At step 206, output of the final output layer of the constructed long and short-range dependency network learning model is used as an indication of classification of the heart rate sounds. The long and short-range dependency network learning model is constructed according to the first aspect of the present disclosure.

The aforementioned steps are only illustrative and other alternatives can also be provided where one or more steps are added, one or more steps are removed, or one or more steps are provided in a different sequence without departing from the scope of the claims herein.

1. A method of constructing long and short-range dependency network learning model, the method comprising:
obtaining heart sound data comprising a plurality of audio files;
preprocessing the plurality of audio files;
extracting Mel-Frequency Cepstral Coefficients (MFCCs) from the preprocessed plurality of audio files;
restructuring extracted MFCCs into an input layer of the long and short-range dependency network learning model;
implementing two or more LSTM network layers with a dropout; and
employing a softmax activation function to a final output layer.

2. The method according to claim 1, wherein the preprocessing comprises at least one of:
sampling the plurality of audio files at a first frequency range;
segmenting the plurality of audio files into compressed frames;
encoding the segmented audio files into a numerical format.

3. The method according to claim 1, wherein the preprocessing further comprises arranging a consistent duration for each audio file from the plurality of audio files.

4. The method according to claim 2, wherein the first frequency range is from 20,000 up to 250,000 Hz.

5. The method according to claim 1, wherein the heart sound data is obtained from a database.

6. The method according to claim 1, wherein the heart sound data is obtained in real-time.

7. The method according to claim 1, wherein MFCCs extraction comprises at least one of the following:
computing 25 MFCCs;
calculating a mean of MFCCs over a time axis;
computing spectral features of MFCCs.

8. The method according to claim 1, wherein the spectral features comprises at least one of selected from a chroma, a mel-spectrogram.

9. The method according to claim 1, wherein the long and short-range dependency network learning model is a transformer model.

10. The method according to claim 1, wherein the long and short-range dependency network learning model is a Long Short-Term Memory (LSTM) network.

11. The method according to claim 1, wherein the two or more LSTM network layers are further implemented with a recurrent dropout.

12. The method according to claim 1 further comprising:
wrapping the two or more LSTM network layers into two or more bidirectional LSTM network layers; and
applying plurality of dense layers with Rectified Linear Unit (ReLU) activation to bidirectional LSTM network layers.

13. The method according to claim 1 further comprising optimizing the constructed long and short-range dependency network learning model with an Adam optimizer.

14. The method according to claim 1 further comprising calculating a loss using categorical cross-entropy.

15. The method of classifying heart rate sound data, the method comprising:
capturing from a patient heart rate sounds to be classified;
inputting the captured heart rate sounds to the input layer of the constructed long and short-range dependency network learning model; and
using output of the final output layer of the constructed long and short-range dependency network learning model as an indication of classification of the heart rate sounds;
wherein the long and short-range dependency network learning model is constructed according to claim 1.

16. The method according to claim 15, wherein the heart sound data is classified to at least one category selected from: normal, murmur, extrasystole, and artifact.

17. The method according to claim 15, wherein the heart rate sounds are captured from the patient in real-time.
